(19) United States
(12) Patent Application Publication
(10) Pub. No.: US 2026/0102096 A1
(43) Pub. Date: Apr. 16, 2026

(54) HEALTH DIAGNOSTIC DEVICE AND METHOD

(71) Applicants: Rose Zerr, Robbinsville, NJ (US); Heather Maerten, Maple Shade, NJ (US)

(72) Inventors: Rose Zerr, Robbinsville, NJ (US); Heather Maerten, Maple Shade, NJ (US)

(21) Appl. No.: 19/323,438

(22) Filed: Sep. 9, 2025

Related U.S. Application Data
(60) Provisional application No. 63/706,885, filed on Oct. 14, 2024.

(51) Int. Cl. A61B 5/282

(52) U.S. Cl. CPC A61B 5/282; A61B 2503/04

(57) ABSTRACT

A health diagnostic device and method of using the same are provided. The device may include a placement body having: an upper torso region configured to be placed on or proximate to the upper torso of a user; a lower torso region configured to be placed on or proximate to the user's lower torso; a right torso region configured to be placed on or proximate to the user's right side torso; a left torso region that to be placed on or proximate to the user's left side torso; a top side surface and an opposing bottom side surface in which the bottom side surface configured to contact the skin of the user's torso. A plurality of electrodes may be coupled to the placement body and may contact the skin of the user's body which may be coupled to a lead line of an electrocardiogram machine or the like.

### CROSS REFERENCE TO RELATED APPLICATIONS

This application claims priority to and the benefit of the filing date of U.S. Provisional Application No. 63/706,885, filed on Oct. 14, 2024, entitled “HEALTH DIAGNOSTIC DEVICE”, which is hereby incorporated by reference in its entirety.

### FIELD OF THE INVENTION

This patent specification relates to the field of health diagnostic devices. More specifically, this patent specification relates to a device which may be placed on an individual to correctly place a plurality of diagnostic electrodes on the individual, such as which may be used for an electrocardiogram (EKG or ECG).

### BACKGROUND

There are many problems related to obtaining electrocardiogram (EKG or ECG), and other health diagnostic tests on infants, toddlers, and even older individuals. Most tests require the precise placement of a plurality of electrodes and lead lines on the individual. This can be especially difficult with infants and toddlers as they are constantly moving and pulling off the wires. Additionally, the placement of a plurality of individual electrodes requires significant training and experience. Unfortunately, even well trained, experienced technicians have approximately a 2% placement error rate that can lead to lost time and misdiagnoses.

Therefore, a need exists for novel health diagnostic devices which are able to reduce error and increase convenience when placing electrodes on the body of an individual.

### BRIEF SUMMARY OF THE INVENTION

A health diagnostic device and method are provided. The device may include a placement body having one or more, such as a plurality, of electrodes embedded in the placement body. Preferably, the method may include placing the placement body of the device on a user's, e.g., infant/child's, chest or torso so that the plurality of electrodes of the device can be placed on the user's torso in one application so that a facility's existing EKG machine lead lines or other health diagnostic device lead lines to obtain an electrocardiogram or other health diagnostic test results.

In some embodiments, the health diagnostic device may include a placement body and a plurality of electrodes that may be coupled to a plurality of locations on the placement body. The placement body may include: an upper torso region adapted to be placed on an upper portion of a torso of a user; a lower torso region adapted to be placed on a lower portion of the torso of the user; a right torso region adapted to be placed on a right side portion of the torso of the user; a left torso region adapted to be placed on a left side portion of the torso of the user torso of the user. The placement body may further include a top side surface and an opposing bottom side surface, and the bottom side surface may be adapted to contact the skin of the user's torso. Each electrode may include a conductive element coupled to a respective lead coupling element so that the conductive element and lead coupling element are in electronic communication. The conductive element of each electrode may be coupled to the bottom side surface and may be configured to contact the skin of the user and conduct electrical activity of the user's body to the lead coupling element when the bottom side surface is placed on the skin of the user's torso. The lead coupling element may be coupled to the top side surface and may be used to couple a lead line of an electrocardiogram (EKG or ECG) machine and other health diagnostic test machines to each electrode.

In some embodiments, a health diagnostic method is provided which may be used for the placement of a plurality of electrode conducting elements of a plurality of electrodes into contact with a torso of a user using a health diagnostic device. Preferably, the method may be performed with a health diagnostic device as described herein by placing the bottom side surface of the placement body on the skin of the user's torso thereby placing, in one application, the plurality of electrodes in the proper position on the user's torso so that the existing facility's existing EKG machine or the like can be connected to the electrodes of the placement body to obtain an electrocardiogram or like test results of the user. Preferably, the method may include placing the bottom side surface of the placement body on the skin of the torso of the user so that the upper torso region is placed on the upper portion of the torso of the user, so that the lower torso region is placed on the lower portion of the torso of the user, so that the right torso region is placed on the right side portion of the torso of the user, and so that the left torso region is placed on the left side portion of the torso of the user. The method may further include the step of coupling or connecting one or more lead lines to the lead coupling element of one or more electrodes so that a single lead line is coupled to the lead coupling element of a single electrode. Optionally, the lead lines may be coupled to the lead coupling elements before the placement body is brought into contact with the user's torso. The method may further include the step of obtaining or recording electrocardiogram or other health diagnostic test results using the electrical activity of the user's body that is picked up via the conductive element of the one or more electrodes.

Numerous objects, features and advantages of the present invention will be readily apparent to those of ordinary skill in the art. Some example objects of the present invention are listed below.

One object of the present invention is to provide a health diagnostic device and method that is unlike any other product or method because they allow a single unit (e.g., silicone material with embedded electrodes) to be placed on the infant/child or other individual to precisely obtain an electrocardiogram instead of individually placing thirteen different electrodes. It is faster, more accurate, less traumatic for the child or other aged individual.

Another object is to provide a health diagnostic device and method utilizing relatively few parts/components that can be used in many healthcare areas without requiring extensive training on how and where to place the electrodes, just place the bottom side surface on the chest or torso. The consistency and accuracy the present invention provides is the ultimate goal to identify any cardiac or other electrical activity detectable abnormality or life-threatening rhythm.

### BRIEF DESCRIPTION OF THE DRAWINGS

Some embodiments of the present invention are illustrated as an example and are not limited by the figures of the accompanying drawings, in which like references may indicate similar elements and in which:

FIG. 1 depicts a top plan view of an example of a health diagnostic device according to various embodiments described herein.

FIG. 2 illustrates a top plan view of an example of a health diagnostic device that includes a coversheet according to various embodiments described herein.

FIG. 3 shows a bottom plan view of an example of a health diagnostic device according to various embodiments described herein.

FIG. 4 depicts another top plan view of an example of a health diagnostic device according to various embodiments described herein.

FIG. 5 illustrates a sectional, through line 5-5 shown in FIG. 1, elevation view of a portion of an example health diagnostic device according to various embodiments described herein.

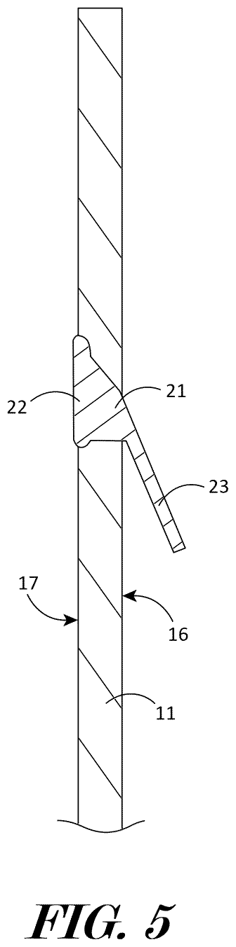

FIG. 6 shows a top plan view of an example of a health diagnostic device placed on the torso of a user according to various embodiments described herein.

FIG. 7 depicts a block diagram of an example of a health diagnostic method using a health diagnostic device according to various embodiments described herein.

FIG. 8 illustrates a perspective view of an example of a health diagnostic device coupled to a plurality of lead lines about to be placed on the torso of a user according to various embodiments described herein.

FIG. 9 shows a perspective view of an example of a health diagnostic device coupled to a plurality of lead lines being placed on the torso of a user according to various embodiments described herein.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

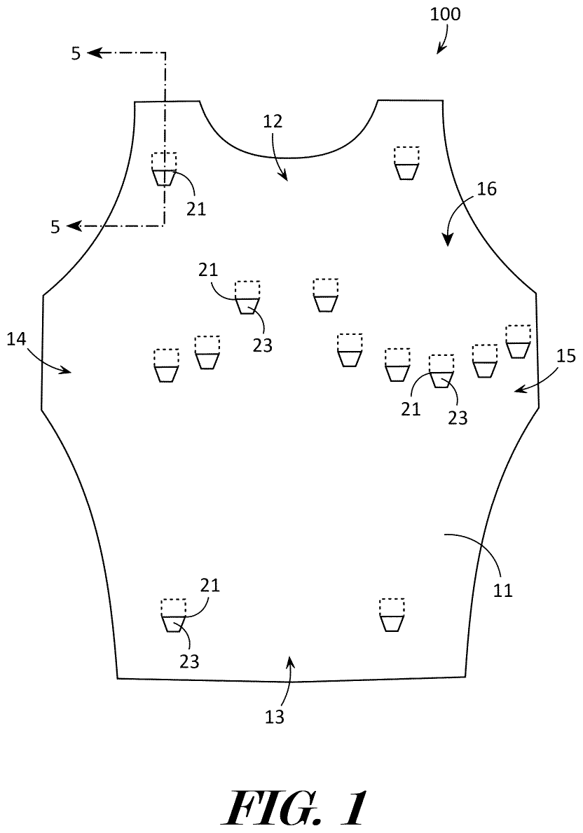

For purposes of description herein, the terms “upper,” “lower,” “left,” “right,” “rear,” “front,” “side,” “vertical,” “horizontal,” and derivatives thereof shall relate to the invention as oriented in FIG. 1. However, one will understand that the invention may assume various alternative orientations and step sequences, except where expressly specified to the contrary. Therefore, the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Although the terms “first,” “second,” etc. are used herein to describe various elements, these elements should not be limited by these terms. These terms are only used to distinguish one element from another element. For example, the first element may be designated as the second element, and the second element may be likewise designated as the first element without departing from the scope of the invention.

As used in this application, the term “about” or “approximately” refers to a range of values within plus or minus 20% of the specified number. Additionally, as used in this application, the term “substantially” means that the actual value is within about 10% of the actual desired value, particularly within about 5% of the actual desired value and especially within about 1% of the actual desired value of any variable, element or limit set forth herein.

A new health diagnostic device and method are discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

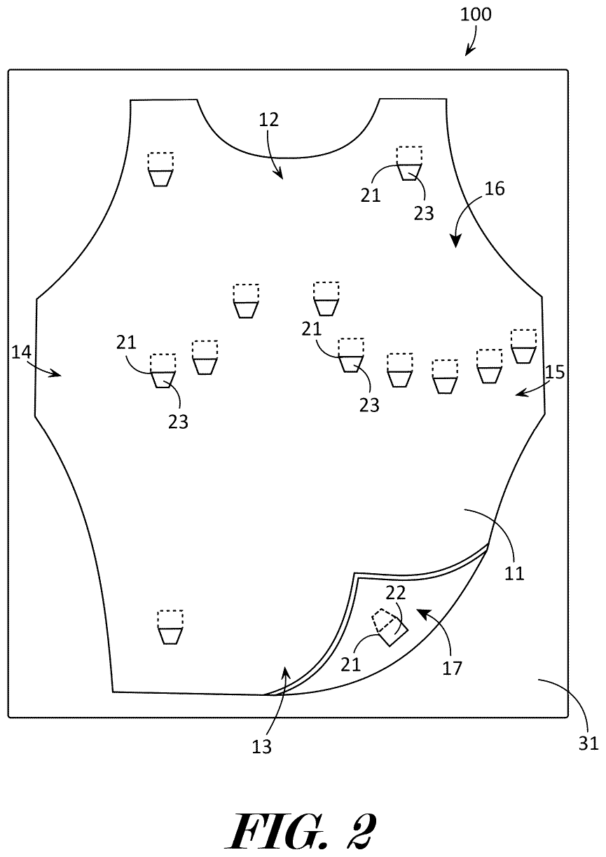

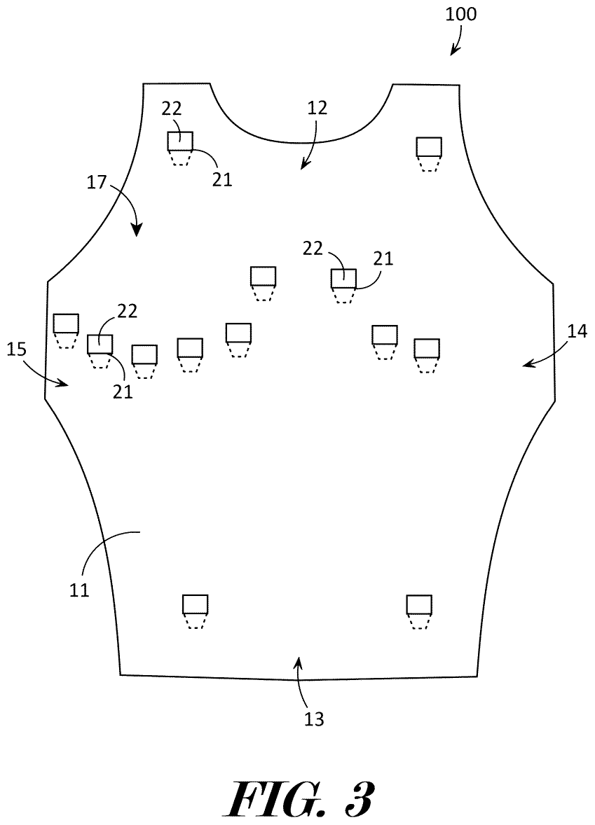

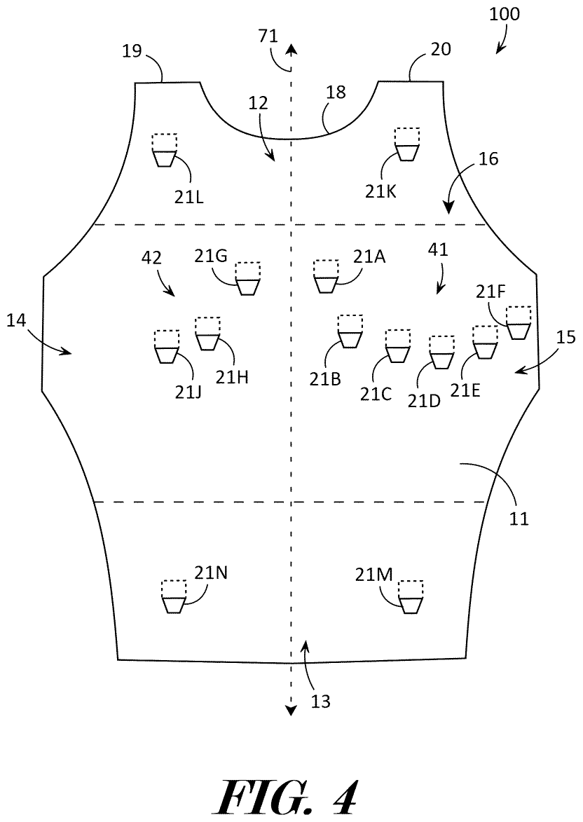

The present invention will now be described by example and through referencing the appended figures representing preferred and alternative embodiments. FIG. 1-4 illustrate an example of a health diagnostic device (“the device”) 100 according to various embodiments. In some embodiments, the device 100 may comprise a placement body 11 and a plurality of electrodes 21 that may be coupled to a plurality of locations on the placement body 11. Preferably the placement body 11 may comprise a soft flexible material, such as silicone. The placement body 11 may comprise: an upper torso region 12 that may be configured or adapted to be placed on or proximate to an upper portion of a torso 202 of a user 200; a lower torso region 13 that may be configured or adapted to be placed on or proximate to a lower portion of the torso 203 of the user 200; a right torso region 14 that may be configured or adapted to be placed on or proximate to a right side portion of the torso 204 of the user 200; and a left torso region 15 that may be configured or adapted to be placed on or proximate to a left side portion of the torso 205 of the user 200. The placement body 11 may comprise a top side surface 16 and an opposing bottom side surface 17, and the bottom side surface 17 may be configured to contact the skin 206 of the user's torso 201. Each electrode 21 may comprise a conductive element 22 and a lead coupling element 23. The conductive element 22 may be coupled to the bottom side surface 17 and may be configured to contact the skin 206 of the torso 201 and conduct electrical activity of the user's 200 body to the lead coupling element 23 when the bottom side surface 17 is placed on the skin 206 of the user's torso 201. The lead coupling element 23 may be coupled to the top side surface 16 and may be used to couple a lead line 400 of an electrocardiogram (EKG or ECG) machine and other health diagnostic test machines to the electrode 21. Preferably, the device 100 may be used by placing the bottom side surface 17 of the placement body 11 on the skin of the user's torso 201 thereby placing, in one application, the plurality of electrodes 21 in the proper position on the user's torso 201 so that the existing facility's existing EKG machine or the like lead lines 400 can be connected to the electrodes 21 of the placement body 11 to obtain an electrocardiogram or the like test results.

The device 100 may comprise a placement body 11 that may be configured in any size and shape. In preferred embodiments, the placement body 11 may be configured generally with the shape of the front half of a vest that may be placed on top of a user's torso 201 while they are in a supine or partially supine position. In some embodiments, the upper torso region 12 may comprise a neck depression 18, left shoulder extension 19, and a right shoulder extension 20, and the neck depression 18 may be positioned between the left shoulder extension 19 and the right shoulder extension 20. Preferably, a neck depression 18 may comprise a cutout, such as which may be used to form an arc or neckline shape that may be positioned proximate to the neck or the user 200 while the left shoulder extension 19 and right shoulder extension 20 may extend away from the neck depression 18 so that they may be positioned proximate to, e.g., draped over, the left shoulder and right shoulder, respectively, of the user 200. In still further embodiments, the placement body 11 may be configured as a shirt, vest, band, or other physical configuration suitable for contacting the torso 201 of a user 200.

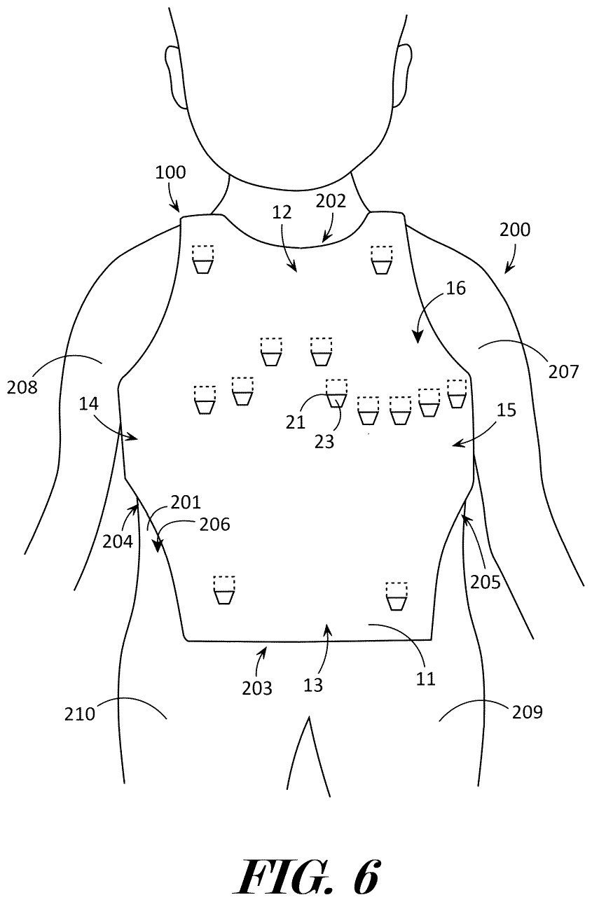

In some embodiments, the placement body 11 may comprise: an upper torso region 12 that may be configured to be placed on or proximate to the upper torso 202 of a user 200; a lower torso region 13 that may be configured to be placed on or proximate to the lower torso 203 of the user 200; a right torso region 14 that may be configured to be placed on or proximate to the right side torso 204 of the user 200; and a left torso region 15 that may be configured to be placed on or proximate to the left side torso 205 of the user 200. Preferably, and as perhaps best shown in FIGS. 6, 8, and 9, the right torso region 14 and left torso region 15 may be centrally located on the placement body 11 so that they are between the upper torso region 12 and the lower torso region 13. The placement body 11 may comprise a center line 71 that preferably may bisect the upper torso region 12 and the lower torso region 13, and the center line 71 may separate the right torso region 14 and left torso region 15.

In some embodiments, the placement body 11 may be configured in a relatively smaller shape for use with infants. In further embodiments, the placement body 11 may be configured in a relatively larger shape for use with toddlers. Generally, the device 100 may be configured with a placement body 11 of a plurality of sizes that may be scaled up and down to accommodate users 200 having a plurality of sized torsos. For example, the placement body 11 may generally resemble the size of clothing sizes, such as P (Preemie): Up to 5 pounds; NB (Newborn): 5 to 8 pounds; 3 months: 8 to 12 pounds; 6 months: 12 to 16 pounds; 9 months: 16 to 20 pounds; 12 months: 20 to 24 pounds; 18 months: 24 to 28 pounds; 24 months: 28 to 30 pounds; 2T: 29 to 32 pounds; 3T: 32 to 35 pounds; 4T: 35 to 39 pounds; and 5T: 39 to 43 pounds. It should be understood that the placement body 11 may be made in larger sizes to accommodate the torsos of children, teenagers, and adults.

The placement body 11 may comprise a top side surface 16 and an opposing bottom side surface 17, and the bottom side surface 17 may be configured to contact the skin of the user's torso 201. Preferably, the placement body 11 may comprise a soft flexible material, such as silicone, that may also be hypoallergenic and suitable for contact with human skin. In further embodiments, the bottom side surface 17 may comprise any other flexible material that is non-conductive to electricity and which is suitable for contacting human skin. In some embodiments, the placement body 11 may comprise a sheet of flexible material, such as a sheet of silicone, polymer sheeting, such as Polypropylene (PP), Medium Density Polyethylene (MDPE), Linear Low Density Polyethylene (LLDPE), Low Density Polyethylene (LDPE), High Density Polyethylene (HDPE), any other polyethylene, Polyvinyl chloride, or any other flexible polymer. In still further embodiments, the placement body 11 may comprise a flexible material that may include fabrics of natural and/or synthetic materials such as, for example, nylon, satin, spandex, cotton, silk, spandex, and polyester or blends thereof, woven materials such as felt, non-woven materials including non-woven spunbond or carded webs of polypropylene, polyethylene, nylon, polyester, a non-woven web of cellulosic fibers, textile fibers such as rayon fibers, or a blend of cellulosic and textile fibers; or melt blown thermoplastic fibers, such as macro fibers or micro fibers of polypropylene, polyethylene, polyester or other thermoplastic materials or mixtures of such thermoplastic macro fibers or micro fibers with cellulosic, pulp or textile fibers, and natural fibers (e.g., wood or cellulose).

The device 100 may comprise a plurality of electrodes 21 that may be coupled to the placement body 11. An electrode 21 may pick up electrical activity through the skin 206 that is transmitted throughout the body, such as which may be used by an electrocardiogram (EKG or ECG) machine and other health diagnostic test machines. Each electrode 21 may comprise a conductive element 22 and a lead coupling element 23. The conductive element 22 may be coupled to the bottom side surface 17 and may be configured to contact the skin 206 of a user's torso 201 and conduct electrical activity of the user's 200 body to the lead coupling element 23.

A conductive element 22 may be configured to contact the skin 206 of a user's torso 201 by being coupled to the placement body 11 so that the conductive element 22 is flush with the bottom side surface 17, and more preferably so that the conductive element 22 protrudes away (above or below depending on orientation of the placement body 11) from the bottom side surface 17. Conductive elements 22 may be configured in any shape and size suitable for picking up electrical activity of the body of a user 200. For example, a conductive element may comprise a round shape, square shape, triangular shape, etc., configured to have a surface area of approximately 22.225 millimeters (mm) (0.875 inches (in) square, configured as a small tab electrode measuring approximately 12 mm×32.5 mm (0.5 in×1.281 in), or configured with any other surface area commonly used in skin contacting electrodes. Conductive elements 22 may be made from conductive materials like metals, conductive polymers, or conductive fabrics. Electrodes 21 may comprise “wet” and/or “dry” electrodes. Wet electrodes often use an electrolyte gel, sometimes containing silver/silver chloride (Ag/AgCl), to improve conductivity and reduce skin-electrode impedance. Dry electrodes, on the other hand, are made of solid conductive materials and rely on direct skin contact, sometimes with the help of a sweat layer.

The lead coupling element 23 may be coupled to the top side surface 16 and may be used to couple a lead line 400 of an electrocardiogram (EKG or ECG) machine and other health diagnostic test machines to the electrode 21. Lead coupling element 23 may be made from conductive materials like metals, conductive polymers, or conductive fabrics. Optionally a conductive element 22 and a lead coupling element 23 may be integrally formed together. A lead coupling element 23 may comprise a length or piece of conductive material that a lead clip 401 of a lead line 400 to be coupled to. In preferred embodiments, the lead coupling element 23 of each electrode 21 may extend away from the top side surface to facilitate the ability of a lead line 400 to be coupled to the lead coupling element 23, such as by being plugged into, clamped on, or otherwise coupled. For example, a lead coupling element 23 may extend away from the top side surface to facilitate the ability of a lead clip 401 of a lead line 400 to be clamped on the lead coupling element 23. A lead clip 401, also known as a universal adapter clip or alligator clip, is a device used to connect EKG and other electrodes to the cables of a patient monitor or other diagnostic equipment. These clips 401 are designed to accommodate different electrode types and cable pin sizes, ensuring a secure and reliable connection for accurate EKG readings.

The device 100 may comprise a plurality of electrodes 21 that may be positioned anywhere on the placement body 11. A plurality of electrodes 21 may comprise at least two electrodes 21, and more preferably more than two electrodes 21, such as three, five, ten, fifteen, etc., electrodes 21. In preferred embodiments, the device 100 may comprise thirteen electrodes 21 that may be positioned on the placement body 11 so that they may be positioned in contact with the skin 206 across a user's torso 201, as shown in FIGS. 6, 8, and 9, when the bottom side surface 17 is placed in contact with the skin of the user's torso 201.

In some embodiments, the plurality of electrodes 21 may comprise a first set 41 of electrodes 21, and the first set 41 of electrodes 21 may be positioned proximate to the left torso region 13, optionally by being positioned in the left torso region 13 partway between the upper torso region 12 and the lower torso region 13. In preferred embodiments, a first set 41 of electrodes 21 may comprise one or more of: a first electrode 21A configured as a V2 electrode; a second electrode 21B configured as a V3 electrode; a third electrode 21C configured as a V4 electrode; a fourth electrode 21D configured as a V5 electrode; a fifth electrode 21E configured as a V6 electrode; and a sixth electrode 21F configured as a V7 electrode. In further preferred embodiments, a first set 41 of electrodes 21 may be arranged generally in an arc shape that starts proximate to the center line 71 and extends away from the center line 71, and the arc shape may be convex curved away from the upper torso region 12 and towards the lower torso region 13.

In some embodiments, the plurality of electrodes 21 may comprise a second set 42 of electrodes 21, and the second set 42 of electrodes 21 may be positioned proximate to the right torso region 14, optionally by being positioned in the right torso region 14 partway between the upper torso region 12 and the lower torso region 13. Preferably, the second set 42 of electrodes 21 may be positioned relatively closer to the right torso region 14 than the first set 41 of electrodes 21 is positioned. In preferred embodiments, a second set 42 of electrodes 21 may comprise one or more of: a seventh electrode 21G configured as a V1 electrode; an eight electrode 21H configured as a V3R electrode; and a ninth electrode 21J configured as a V4R electrode. In further preferred embodiments, a second set 42 of electrodes 21 may be arranged generally in an arc shape that starts proximate to the center line 71 and extends away from the center line 71, and the arc shape may be generally convex curved away from the upper torso region 12 and towards the lower torso region 13, such as by being generally convex curved away from a right arm electrode 21L and towards a left leg electrode 21M.

In some embodiments, the plurality of electrodes 21 may comprise a left arm electrode 21K and a right arm electrode 21L. Preferably, the left arm electrode 21K may be positioned proximate to the left shoulder extension 19, and the right arm electrode 21L may be positioned proximate to the right shoulder extension 20. A left arm electrode 21K may be configured as a LA electrode, and a right arm electrode 21L may be configured as a RA electrode.

In some embodiments, the plurality of electrodes 21 may comprise a left leg electrode 21M and a right leg electrode 21N. Preferably, the left leg electrode 21M may be positioned proximate in the lower torso region 13 so that the leg electrode 21M and left arm electrode 21K are on opposite sides of the first set 41 of electrodes 21, and the right leg electrode 21N may be positioned in the lower torso region 13 so that the right leg electrode 21N and right arm electrode 21L are on opposite sides of the second set 42 of electrodes 21. A left leg electrode 21M may be configured as a LL electrode, and a right leg electrode 21N may be configured as a RL electrode.

In some embodiments, the device 100 may comprise a coversheet 31 which may be removably coupled to the bottom side surface 17 and/or conductive elements 22 and which may be removed prior to placing the bottom side surface 17 into contact with the skin of a user's torso 201. Optionally, a coversheet 31 may comprise a paper or plastic-based film sheet.

In some embodiments, the device 100 may comprise one or more fastening elements which may be configured to help secure the device 100 to a user's torso 201 and to maintain its position during testing. For example, the device 100 may comprise fastening elements that may include plastic, fabric, silicone, or other flexible material fasteners, drawstrings, straps, etc. which may wrap around a user's torso 201. As another example, fastening elements may comprise skin safe adhesive.

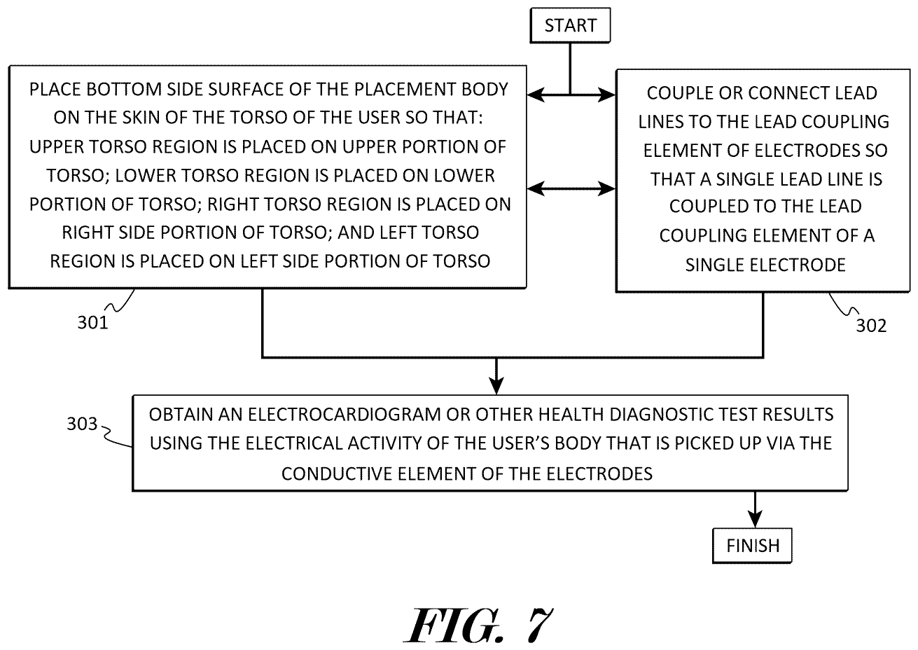

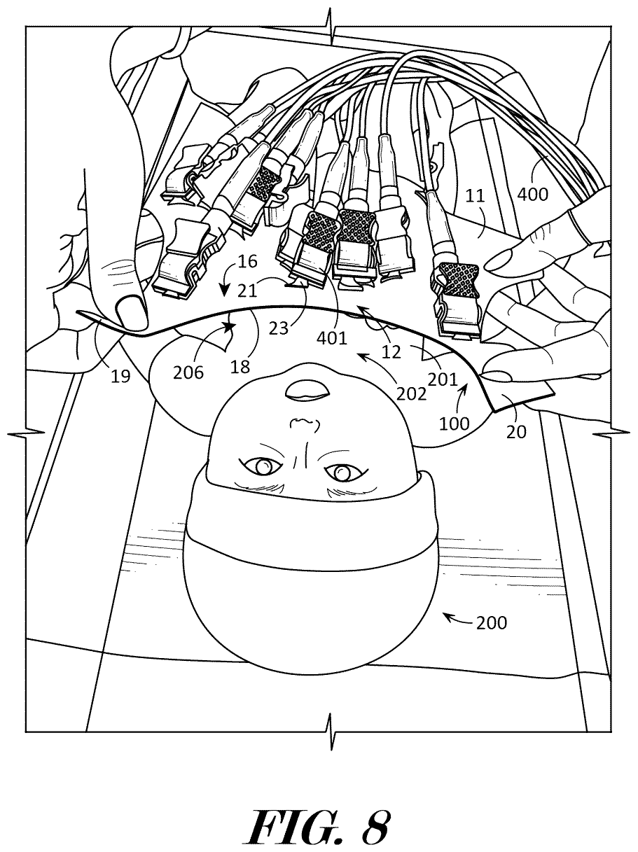

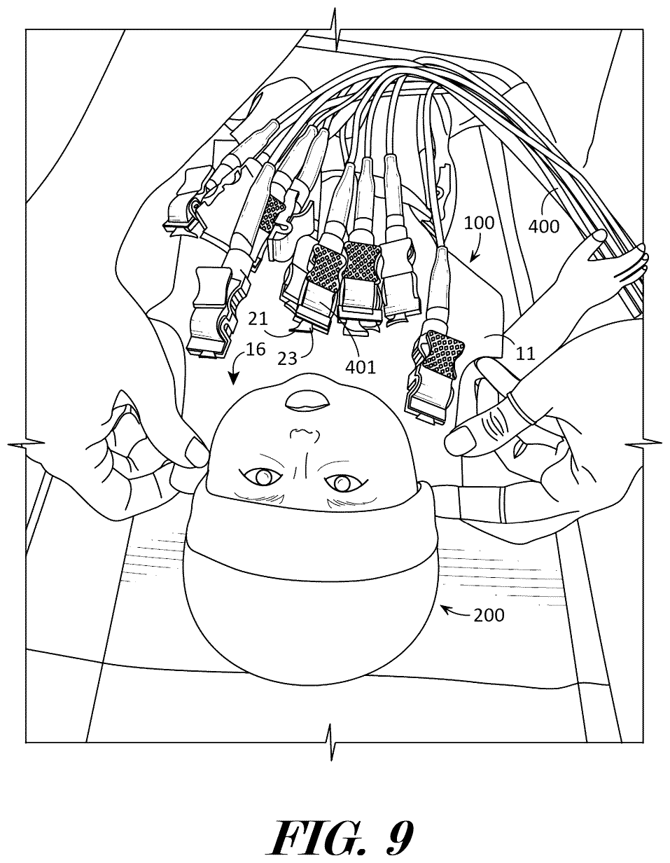

Referring to FIG. 7, a health diagnostic method 300 (“the method 300”) is provided which may be used for the placement of a plurality of electrode conducting elements 22 of a plurality of electrodes 21 into contact with a torso 201 of a user 200 using a health diagnostic device 100 as perhaps best shown in FIGS. 8 and 9. Preferably, the method 300 may be performed with a device 100 by placing the bottom side surface 17 of the placement body 11 on the skin 206 of the user's torso 201 thereby placing, in one application, the plurality of electrodes 21 in the proper position on the user's torso 201 so that the existing facility's existing EKG machine or the like can be connected to the electrodes 21 of the placement body 11 to obtain an electrocardiogram or like test results of the user 200.

In preferred embodiments, the method 300 may be performed using a device 100 that comprises a placement body 11 that includes: an upper torso region 12 adapted to be placed on an upper portion of the torso 202 of the user 200; a lower torso region 13 adapted to be placed on a lower portion of the torso 203 of the user 200; a right torso region 14 adapted to be placed on a right side portion of the torso 204 of the user 200; a left torso region 15 adapted to be placed on a left side portion of the torso 205 of the torso of the user 200; a top side surface 16; and a bottom side surface 17. The bottom side surface 17 may be adapted to contact skin 206 of the torso 201 of the user 200. A plurality of electrodes 21 may be coupled to the placement body 11, and each electrode 21 of the plurality of electrodes 21 may comprise a conductive element 22 coupled to a respective lead coupling element 23. The lead coupling element 23 of each electrode 21 of the plurality of electrodes 21 may be coupled to the top side surface 16. The conductive element 22 of each electrode 21 of the plurality of electrodes 21 may be coupled to the bottom side surface 17 and may be configured to contact the skin 206 of the torso 201 of the user 200 to conduct electrical activity of the torso 201 of the user 200 to the lead coupling element 23 that each conductive element 22 of each electrode 21 is respectively coupled to when the bottom side surface 17 is placed on the skin 206 of the torso 201 of the user 200.

In some embodiments, the method 300 may comprise step 301 in which the bottom side surface 17 of the placement body 11 may be placed on the skin 206 of the torso 201 of the user 200 so that the upper torso region 12 is placed on the upper portion of the torso 202 of the user 200, so that the lower torso region 12 is placed on the lower portion of the torso 203 of the user 200, so that the right torso region 14 is placed on the right side portion of the torso 204 of the user 200, and so that the left torso region 15 is placed on the left side portion of the torso 205 of the user 200.

In some embodiments, the method 300 may use a device 100 in which the plurality of electrodes 21 comprises a first set 41 of electrodes 21, and the first set 41 of electrodes 21 may be positioned on the left side portion of the torso 205 of the user when the bottom side surface 17 of the placement body 11 is placed on the skin 206 of the torso 201 of the user 200. In further embodiments, the method 300 may use a device 100 in which the plurality of electrodes 21 comprises a second set 42 of electrodes 21, and the second set 42 of electrodes 21 may be positioned on the right side portion of the torso 204 of the user 200 when the bottom side surface 17 of the placement body 11 is placed on the skin 206 of the torso 201 of the user 200.

In some embodiments, the method 300 may use a device 100 in which the plurality of electrodes 21 comprises a left arm electrode 21K and a right arm electrode 21L. The left arm electrode 21K may be positioned proximate to a left arm 207 of the user 200 when the bottom side surface 17 of the placement body 11 is placed on the skin 206 of the torso 201 of the user 200, and the right arm electrode 21L may be positioned proximate to a right arm 208 of the user 200 when the bottom side surface 17 of the placement body 11 is placed on the skin 206 of the torso 201 of the user 200.

In some embodiments, the method 300 may use a device 100 in which the plurality of electrodes 21 comprises left leg electrode 21M and a right leg electrode 21N. The left leg electrode 21M may be positioned proximate to a left leg 209 of the user 200 when the bottom side surface 17 of the placement body 11 is placed on the skin 206 of the torso 201 of the user 200, and the right leg electrode 21N may be positioned proximate to a right leg 210 of the user 200 when the bottom side surface 17 of the placement body 11 is placed on the skin 206 of the torso 201 of the user 200.

In some embodiments, the method 300 may comprise step 302 in which one or more lead lines 400 may be coupled or connected to the lead coupling element 23 of one or more electrodes 21 so that a single lead line 400 is coupled to the lead coupling element 23 of a single electrode 21. Optionally, step 302 may be performed before step 301 so that the lead lines 400 may be coupled to the lead coupling elements 23 before the placement body 11 is brought into contact with the user's torso 201.

After the one or more lead lines 400, such as the lead lines 400 of a facility's existing EKG machine or other health diagnostic device that utilizes electrical activity of a person's body, are coupled or connected to the lead coupling element 23 of one or more electrodes 21 and the placement body 11 is brought into contact with the user's torso 201, an electrocardiogram or other health diagnostic test results may be obtained using the electrical activity of the user's body that is picked up via the conductive element 22 of the one or more electrodes 21 in step 303.

While some exemplary shapes and sizes have been provided for elements of the device 100, it should be understood to one of ordinary skill in the art that the placement body 11, electrodes 21, and any other element described herein may be configured in a plurality of sizes and shapes including “T” shaped, “X” shaped, square shaped, rectangular shaped, cylinder shaped, cuboid shaped, hexagonal prism shaped, triangular prism shaped, or any other geometric or non-geometric shape, including combinations of shapes. It is not intended herein to mention all the possible alternatives, equivalent forms or ramifications of the invention. It is understood that the terms and proposed shapes used herein are merely descriptive, rather than limiting, and that various changes, such as to size and shape, may be made without departing from the spirit or scope of the invention.

Additionally, while some materials have been provided, in other embodiments, the elements that comprise the device 100 may be made from or may comprise durable materials such as aluminum, steel, other metals and metal alloys, wood, hard rubbers, hard plastics, fiber reinforced plastics, carbon fiber, fiberglass, resins, polymers or any other suitable materials including combinations of materials. Additionally, one or more elements may be made from or may comprise durable and slightly flexible materials such as soft plastics, silicone, soft rubbers, or any other suitable materials including combinations of materials. In some embodiments, one or more of the elements that comprise the device 100 may be coupled or connected together with heat bonding, chemical bonding, adhesives, clasp type fasteners, clip type fasteners, rivet type fasteners, threaded type fasteners, other types of fasteners, or any other suitable joining method. In other embodiments, one or more of the elements that comprise the device 100 may be coupled or removably connected by being press fit or snap fit together, by one or more fasteners such as hook and loop type or Velcro® fasteners, magnetic type fasteners, threaded type fasteners, sealable tongue and groove fasteners, snap fasteners, clip type fasteners, clasp type fasteners, ratchet type fasteners, a push-to-lock type connection method, a turn-to-lock type connection method, a slide-to-lock type connection method or any other suitable temporary connection method as one reasonably skilled in the art could envision to serve the same function. In further embodiments, one or more of the elements that comprise the device 100 may be coupled by being one of connected to and integrally formed with another element of the device 100.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. A health diagnostic device, the device including:
a placement body, wherein the placement body comprises: an upper torso region adapted to be placed on an upper portion of a torso of a user; a lower torso region adapted to be placed on a lower portion of the torso of the user; a right torso region adapted to be placed on a right side portion of the torso of the user; a left torso region adapted to be placed on a left side portion of the torso of the user torso of the user; a top side surface; and a bottom side surface, wherein the bottom side surface is adapted to contact skin of the torso of the user; and
a plurality of electrodes coupled to the placement body, wherein each electrode of the plurality of electrodes comprises a conductive element coupled to a respective a lead coupling element, wherein the conductive element of each electrode of the plurality of electrodes is coupled to the bottom side surface and is configured to contact the skin of the torso of the user to conduct electrical activity of the torso of the user to the lead coupling element that each conductive element of each electrode is respectively coupled to when the bottom side surface is placed on the skin of the torso of the user, and wherein the lead coupling element of each electrode of the plurality of electrodes is coupled to the top side surface.

2. The device of claim 1, wherein the placement body comprises a sheet of flexible material.

3. The device of claim 2, wherein the flexible material comprises silicone.

4. The device of claim 1, the lead coupling element of each electrode of the plurality of electrodes extends away from the top side surface.

5. The device of claim 1, wherein the upper torso region comprises a neck depression, left shoulder extension, and a right shoulder extension, and wherein the neck depression is positioned between the left shoulder extension and the right shoulder extension.

6. The device of claim 5, wherein the plurality of electrodes comprises a left arm electrode and a right arm electrode, wherein the left arm electrode is positioned proximate to the left shoulder extension, and wherein the right arm electrode is positioned proximate to the right shoulder extension.

7. The device of claim 1, wherein the plurality of electrodes comprises a first set of electrodes, and wherein the first set of electrodes is positioned proximate to the left torso region.

8. The device of claim 7, wherein the plurality of electrodes comprises a second set of electrodes, and wherein the second set of electrodes is positioned relatively closer to the right torso region than the first set of electrodes is positioned.

9. The device of claim 1, wherein the plurality of electrodes comprises a left leg electrode and a right leg electrode, and wherein the left leg electrode and the right leg electrode are positioned in the lower torso region.

10. A health diagnostic method for the placement of a plurality of electrode conducting elements of a plurality of electrodes into contact with a torso of a user using a health diagnostic device, wherein the device comprises: a placement body, wherein the placement body comprises: an upper torso region adapted to be placed on an upper portion of the torso of the user; a lower torso region adapted to be placed on a lower portion of the torso of the user; a right torso region adapted to be placed on a right side portion of the torso of the user; a left torso region adapted to be placed on a left side portion of the torso of the torso of the user; a top side surface; and a bottom side surface, wherein the bottom side surface is adapted to contact skin of the torso of the user; and a plurality of electrodes coupled to the placement body, wherein each electrode of the plurality of electrodes comprises a conductive element coupled to a respective a lead coupling element, wherein the conductive element of each electrode of the plurality of electrodes is coupled to the bottom side surface and is configured to contact the skin of the torso of the user to conduct electrical activity of the torso of the user to the lead coupling element that each conductive element of each electrode is respectively coupled to when the bottom side surface is placed on the skin of the torso of the user, and wherein the lead coupling element of each electrode of the plurality of electrodes is coupled to the top side surface; and wherein the method comprises the step of:
placing the bottom side surface of the placement body on the skin of the torso of the user so that the upper torso region is placed on the upper portion of the torso of the user, so that the lower torso region is placed on the lower portion of the torso of the user, so that the right torso region is placed on the right side portion of the torso of the user, and so that the left torso region is placed on the left side portion of the torso of the user.

11. The method of claim 10, wherein the placement body comprises a sheet of flexible material.

12. The method of claim 11, wherein the flexible material comprises silicone.

13. The method of claim 10, the lead coupling element of each electrode of the plurality of electrodes extends away from the top side surface.

14. The method of claim 10, wherein the upper torso region comprises a neck depression, left shoulder extension, and a right shoulder extension, and wherein the neck depression is positioned between the left shoulder extension and the right shoulder extension.

15. The method of claim 10, wherein the plurality of electrodes comprises a first set of electrodes, and wherein the first set of electrodes is positioned on the left side portion of the torso of the user when the bottom side surface of the placement body is placed on the skin of the torso of the user.

16. The method of claim 15, wherein the plurality of electrodes comprises a second set of electrodes, and wherein the second set of electrodes is positioned on the right side portion of the torso of the user when the bottom side surface of the placement body is placed on the skin of the torso of the user.

17. The method of claim 10, wherein the plurality of electrodes comprises a left arm electrode and a right arm electrode, wherein the left arm electrode is positioned proximate to a left arm of the user when the bottom side surface of the placement body is placed on the skin of the torso of the user, and wherein the right arm electrode is positioned proximate to a right arm of the user when the bottom side surface of the placement body is placed on the skin of the torso of the user.

18. The method of claim 10, wherein the plurality of electrodes comprises a left leg electrode and a right leg electrode, wherein the left leg electrode is positioned proximate to a left leg of the user when the bottom side surface of the placement body is placed on the skin of the torso of the user, and wherein the right leg electrode is positioned proximate to a right leg of the user when the bottom side surface of the placement body is placed on the skin of the torso of the user.
